(19) United States
(12) Patent Application Publication
(10) Pub. No.: US 2023/0061198 A1
(43) Pub. Date: Mar. 2, 2023

(54) SYSTEM FOR PERFORMING LASER ACUPUNCTURE

(71) Applicant: I SHOU UNIVERSITY, Kaohsiung City (TW)

(72) Inventors: Chih-Yu WANG, Kaohsiung City (TW); Kun-Chan LAN, Kaohsiung City (TW); Che-Chang KUO, Kaohsiung City (TW); Shu-Chen CHANG, Kaohsiung City (TW)

(73) Assignee: I SHOU UNIVERSITY, Kaohsiung City (TW)

(21) Appl. No.: 17/898,041

(22) Filed: Aug. 29, 2022

(30) Foreign Application Priority Data
Aug. 31, 2021 (TW) 110132264

(51) Int. Cl. A61N 5/06; A61N 5/067; A61H 39/02

(52) U.S. Cl. CPC A61N 5/0619; A61N 5/067; A61H 39/02; A61N 2005/0627

(57) ABSTRACT

A system includes a frame, and an image processing unit, a laser unit and an adjusting unit that are mounted on the frame. The image processing unit includes an image capturing device and a processor. The image capturing device captures an image of a body part. The processor generates a control signal based on the image. The laser unit includes a light source and an optical component. The light source emits a laser beam. The adjusting unit is connected to the processor, and interconnects the optical component and the frame. The adjusting unit receives the control signal, and orients, based on the control signal, the optical component to direct the laser beam to acupuncture points on the body part individually.

### CROSS-REFERENCE TO RELATED APPLICATION

This application claims priority of Taiwanese Invention Patent Application No. 110132264, filed on Aug. 31, 2021.

### FIELD

The disclosure relates to a system for performing laser acupuncture.

### BACKGROUND

Traditionally, laser acupuncture is performed manually by a medical practitioner based on his/her expertise and experience, and quality of performance cannot be guaranteed due to practical variabilities (e.g., proficiency of the medical practitioner in using acupoint-finding techniques and differences in body shapes of patients).

A conventional laser acupuncture device emits a laser beam that will be directed to pass through a plurality of preset paths, one at a time, to reach a plurality of desired positions on a body part of a patient, where the desired positions correspond respectively to a plurality of acupuncture points. Using the conventional laser acupuncture device greatly reduces manual operations involved in performing laser acupuncture. However, because the preset paths are fixed, the conventional laser acupuncture device is only suitable for patients with a specific type of body shape.

### SUMMARY

Therefore, an object of the disclosure is to provide a system for performing laser acupuncture on a plurality of acupuncture points on a body part of a subject that can alleviate at least one of the drawbacks of the prior art.

According to the disclosure, the system includes a frame, an image processing unit, a laser unit and an adjusting unit.

The image processing unit is mounted on the frame, and includes an image capturing device and a processor. The image capturing device is configured to capture an image of the body part having the acupuncture points. The processor is configured to generate, based on the image of the body part, a control signal that contains information related to positions respectively of the acupuncture points.

The laser unit is mounted on the frame and is spaced apart from the image processing unit. The laser unit includes a light source and an optical component. The light source is configured to emit a laser beam. The optical component is located in a path, in which the laser beam travels, and is configured to direct the laser beam to a desired position.

The adjusting unit is mounted on the frame. The adjusting unit is electrically connected to the processor, and interconnects the optical component and the frame. The adjusting unit is configured to receive the control signal, and to orient, based on the control signal, the optical component to direct the laser beam to the acupuncture points individually.

### BRIEF DESCRIPTION OF THE DRAWINGS

Other features and advantages of the disclosure will become apparent in the following detailed description of the embodiments with reference to the accompanying drawings, of which:

FIG. 1 is a block diagram illustrating a system for performing laser acupuncture according to an embodiment of the disclosure;

FIG. 2 is a schematic diagram illustrating a process performed by an image processing unit of the system according to the embodiment of the disclosure;

FIGS. 3 and 4 are schematic diagrams cooperatively illustrating ways of orienting an optical component of the system according to the embodiment of the disclosure;

FIG. 5 is a schematic diagram illustrating performing laser acupuncture by using the system according to the embodiment of the disclosure;

FIG. 6 is a schematic diagram illustrating performing laser acupuncture by using the system according to a variant embodiment of the disclosure; and

FIG. 7 is a schematic diagram illustrating an artificial neural network utilized by the system according to the disclosure.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

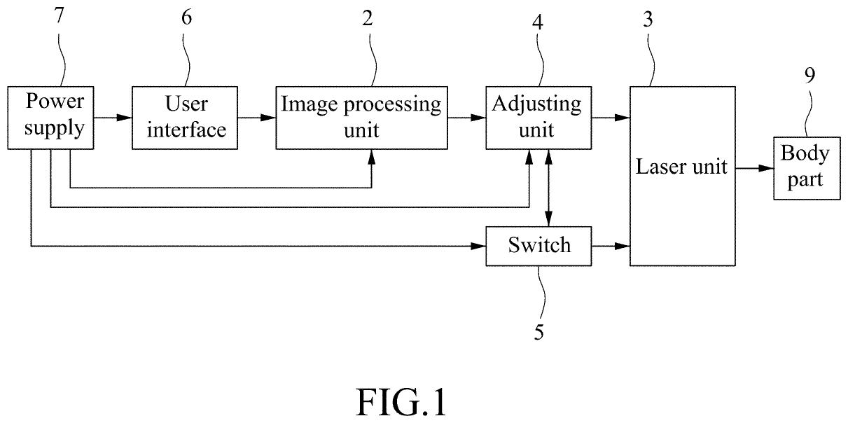

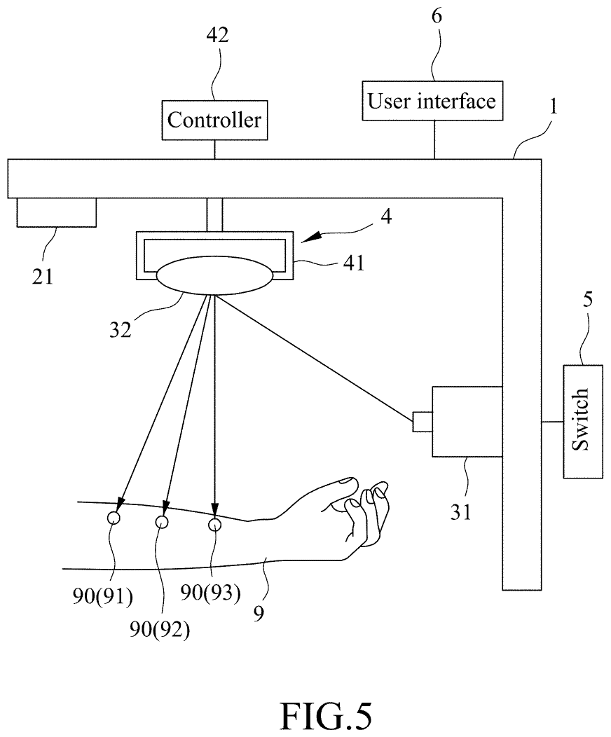

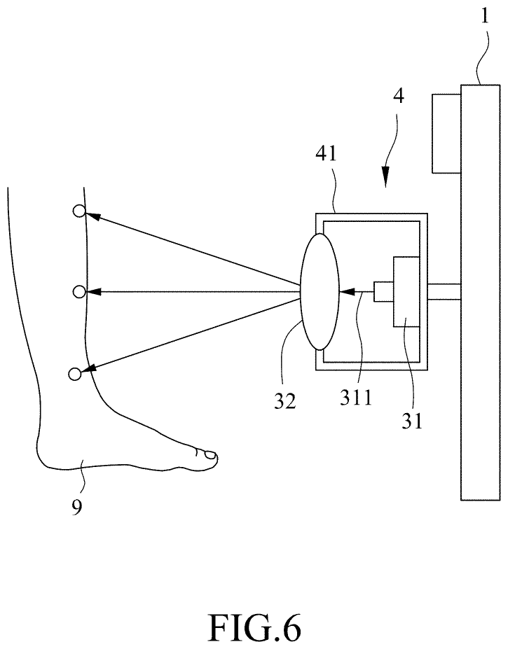

FIG. 1 illustrates an embodiment of a system according to the disclosure for performing laser acupuncture on a plurality of acupuncture points on a body part 9 of a subject (e.g., a patient, not shown). The body part 9 may be an arm (see FIG. 5), a leg (see FIG. 6), etc., but is not limited thereto. It should be noted that for clarity of explanation, only one subject is given as an example hereinafter, and no limitation is placed on conditions (e.g., body shape) of the subject who is subjected to laser acupuncture performed by the system.

The system includes a frame 1 (see FIGS. 5 and 6), an image processing unit 2, a laser unit 3, an adjusting unit 4, a switch 5, a user interface 6 and a power supply 7.

The power supply 7 is electrically connected to the image processing unit 2, the laser unit 3, the adjusting unit 4, the switch 5 and the user interface 6, and is configured to supply electricity to those components. The power supply 7 may be implemented by a storage battery (also known as a rechargeable battery or a secondary cell), or by an electrical grid that provides mains electricity via AC power plugs and sockets.

Referring to FIG. 5, the frame 1 includes two metal rods that are traverse to each other. In this embodiment, the two metal rods are perpendicularly connected to each other and one of the two metal rods extends in a horizontal direction. However, the way of connecting the two metal rods is not limited to the disclosure herein and may vary in other embodiments. The frame 1 is configured to provide a stable foundation for the whole system, especially during performance of laser acupuncture. However, it is worth noting that the size and the shape of the frame 1 are not limited to the disclosure herein and may vary in other embodiments.

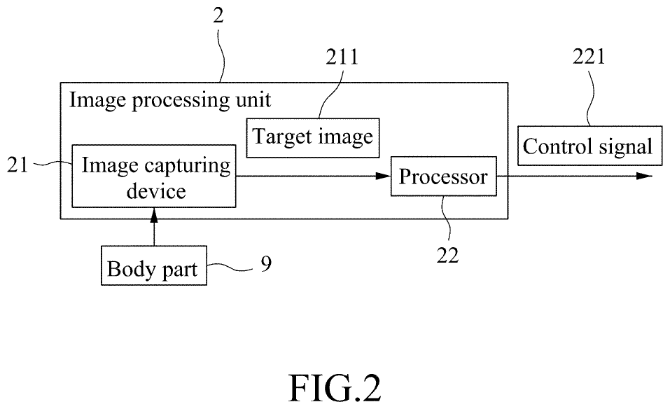

The image processing unit 2 is mounted on the frame 1. Referring to FIG. 2, the image processing unit 2 includes an image capturing device 21 and a processor 22. The image capturing device 21 is configured to capture an image of the body part 9 (hereinafter referred to as a target image 211). The processor 22 is configured to generate, based on the target image 211, a control signal 221 that contains information related to positions respectively of acupuncture points of the body part 9. In this embodiment, the image processing unit 2 is implemented by a mobile phone, the image capturing device 21 and the processor 22 are respectively implemented by a camera and a microcontroller of the mobile phone. However, implementations of the image processing unit 2, the image capturing device 21 and the processor 22 are not limited to what are disclosed herein and may vary in other embodiments. For example, the image processing unit 2 may be implemented by a tablet computer including a camera, a notebook computer including a camera, a depth camera electrically connected to a personal computer, a system on a chip (SoC), or any circuit configurable/programmable in a software manner and/or hardware manner to implement functionalities discussed in this disclosure.

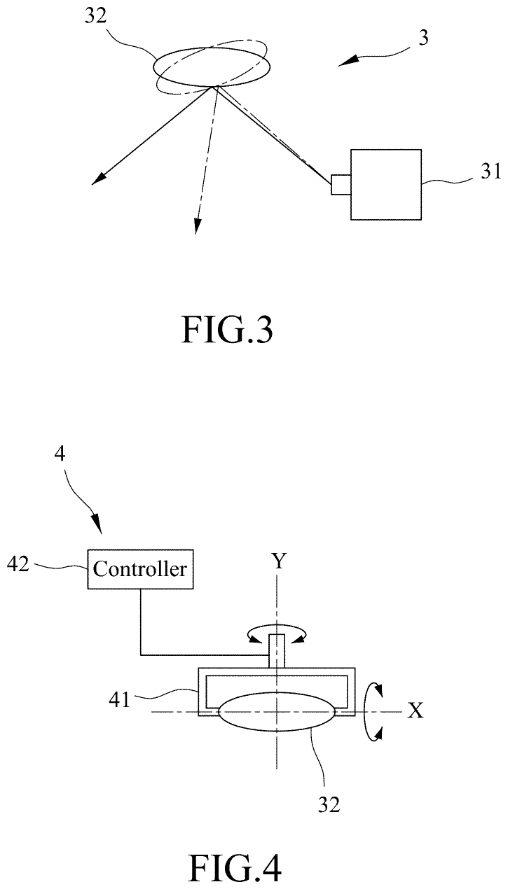

Referring to FIGS. 3, 4 and 5, the laser unit 3 is mounted on the frame 1, and is spaced apart from the image processing unit 2. The laser unit 3 includes a light source 31 and an optical component 32. The light source 31 is mounted on the frame 1, and is configured to emit a laser beam. The optical component 32 is located in a path, along which the laser beam travels, and is configured to direct the laser beam to a desired position. The light source 31 is a laser transmitter that is implemented by a laser diode, a laser fiber or the like. It should be noted that the laser beam emitted by the light source 31 has low power and would not harm human bodies. The optical component 32 may be implemented by a reflector, a refracting lens or the like, but is not limited to what are disclosed herein and may vary in other embodiments. It should be noted that the optical component 32 is illustrated as an ellipsoid as shown in FIGS. 3, 4 and 5, but the shape of the optical component 32 is not limited thereto. In some embodiments, the optical component 32 is a two-axis laser scanning head that includes an X-axis scanning mirror and a Y-axis scanning mirror. In one embodiment, the X-axis and Y-axis scanning mirrors can be implemented by a 2-dimensional Galvo mirror.

The adjusting unit 4 is mounted on the frame 1. The adjusting unit 4 is electrically connected to the processor 22, and interconnects the optical component 32 and the frame 1. The adjusting unit 4 includes a controller 42, and a manipulator 41 that is electrically connected to the controller 42 and that is coupled to the optical component 32. The controller 42 is configured to receive the control signal 221 from the processor 22, and to control the manipulator 41 to orient, based on the control signal 221, the optical component 32 to direct the laser beam to the acupuncture points individually (i.e., to direct the laser beam to one acupuncture point at a time). In this embodiment, the optical component 32 defines a first axis and a second axis that are perpendicular to each other and that interest at a center of the optical component 32. The controller 42 is configured to control the manipulator 41 to rotate the optical component 32 about the first axis of the optical component 32, or to rotate the optical component 32 about the second axis of the optical component 32. The controller 42 may be implemented by a central processing unit (CPU), a microprocessor, a micro control unit (MCU), a system on a chip (SoC), or any circuit configurable/programmable in a software manner and/or hardware manner to implement functionalities discussed in this disclosure.

An example of a method for generating the control signal 221 for orienting the optical component 32 based on the target image 211 is delineated below.

Firstly, a reference object having a top surface, on which a plurality of markers (e.g., stickers) are evenly and densely distributed, is placed under the image capturing device 21. The image capturing device 21 then captures an image (hereinafter referred to as a reference image) of the top surface of the reference object. Specific coordinate sets in a two-dimensional Cartesian coordinate system are assigned respectively to the markers in the reference image. For each of the markers on the top surface of the reference object, the optical component 32 is manually oriented to have a specific orientation in a manner that the laser beam emitted from the light source 31 is directed by the optical component 32 to the marker. Subsequently, for each of the markers, a correspondence relationship between the specific coordinate set of the marker in the reference image and the specific orientation of the optical component 32 (i.e., an angular positon of the optical component 32 on the X-axis and an angular positon of the optical component 32 on the Y-axis) oriented for the marker on the top surface is recorded in a mapping table. Then, a first artificial neural network (ANN) is trained using the specific coordinate sets of the markers as input and the specific orientations corresponding respectively to the specific coordinate sets as output. Since implementation of ANNs is well known to one skilled in the relevant art, detailed explanation of the same is omitted herein for the sake of brevity.

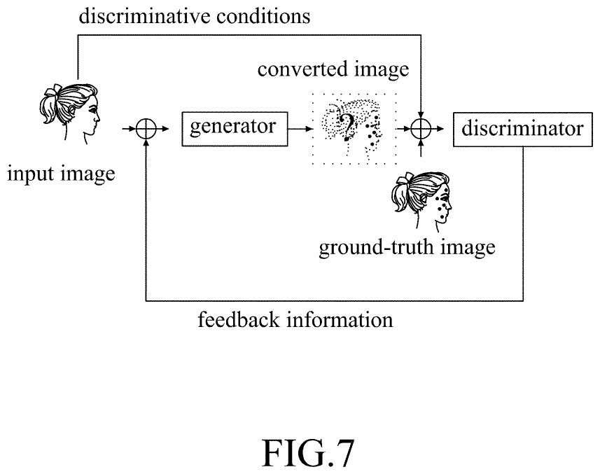

In addition, techniques of paired image-to-image translation (also known as a pix2pix process) using supervised learning is utilized to train a second ANN that is establish under generative adversarial network (GAN) framework and that is configured to identify positions respectively of acupuncture points in an image of a body part of a subject. In particular, referring to FIG. 7, the second ANN includes a generator that is implemented by a U-Net, and a discriminator that is implemented by a Patch-GAN. A plurality of pairs of training images are utilized to train the second ANN. For each pair of training images, one image of the pair of training images contains a body part having no indication on where acupuncture points are located on the body part and serves as an input image, and the other image of the pair of training images contains the same body part having indications respectively indicating where acupuncture points are located on the body part and serves as a ground-truth image. The indications are visual markers. The generator is configured to convert the input image into a converted image. The discriminator is configured to compare the converted image with the ground-truth image under guidance of discriminative conditions that are derived from the input image, and to generate, based on a result of comparison, feedback information that is used for adjusting parameters of the generator. After the generator is adjusted using the feedback information, the generator converts the input image into another converted image, and the discriminator compares said another converted image with the ground-truth image and generates feedback information for further adjusting the parameters of the generator. This process is repeated until the converted image generated by the generator is substantially identical to the ground-truth image.

It is worthy to note that the input image and the ground-truth image are prepared by using the same camera to capture, via a linear polarizing lens, images of the body part having linear polarizing films adhered on acupuncture points respectively under two shooting conditions. Specifically, the linear polarizing lens in one of the shooting conditions is rotated by a preset angle (e.g., 90 degrees) relative to that in the other of the shooting conditions in a manner that the image captured in the one of the shooting conditions contains indications indicating where the acupuncture points are located and the image captured in the other of the shooting conditions contains no such indications. The input image and the ground-truth image are respectively stored in an indication-free image database and an indication-defined image database.

In one embodiment, the ground-truth image is prepared by using the above-mentioned approach, i.e., utilizing the camera to capture, via the linear polarizing lens, an image of the body part having the linear polarizing films adhered on the acupuncture points in a manner that the image thus captured contains indications indicating where the acupuncture points are located. However, the input image is prepared by performing image post-processing on the ground-truth image to obtain an image that contains no such indications.

It should be noted that each of the first ANN and the second ANN may be implemented by one of firmware, software, and any combination thereof. For example, the first ANN and the second ANN may be implemented to be software modules of a program (e.g., an application program stored in the image processing unit 2). Specifically, the software modules contain codes and instructions to carry out specific functionalities, and can be called individually or together to fulfill the method for generating the control signal 221 for orienting the optical component 32 based on the target image 211 of this disclosure.

The above-mentioned modules may be embodied in: executable software as a set of logic instructions stored in a machine- or computer-readable storage medium of a memory such as random access memory (RAM), read only memory (ROM), programmable ROM (PROM), firmware, flash memory, etc.; configurable logic such as programmable logic arrays (PLAs), field programmable gate arrays (FPGAs), complex programmable logic devices (CPLDs), etc.; fixed-functionality logic hardware using circuit technology such as application specific integrated circuit (ASIC), complementary metal oxide semiconductor (CMOS), transistor-transistor logic (TTL) technology, etc.; or any combination thereof.

In this way, when the target image 211 is captured in the same way as the reference image, the processor 22 is capable of utilizing the second ANN to add markers to the target image 211 respectively at the acupuncture points, and utilizing the first ANN to obtain the specific orientations that respectively correspond to the specific coordinate sets of the markers. Thereafter, the processor 22 generates the control signal 221 based on the specific orientations thus obtained, and sends the control signal 221 to the controller 42 of the adjusting unit 4 so as to enable the controller 42 to control the manipulator 41 to orient the optical component 32 such that the laser beam is directed to the acupuncture points, one at a time.

The switch 5 is electrically connected to the light source 31 and the adjusting unit 4. The switch 5 is configured to switch between an on state and an off state. In the on state, the switch 5 enables the light source 31 to emit the laser beam. In the off state, the switch 5 disables the light source 31 so the light source 31 is unable to emit the laser beam. In one embodiment, the switch 5 is implemented to be capable of rapidly switching between the on state and the off state. In one embodiment, the switch 5 is implemented to utilize a multi-vibrator. In one embodiment, the switch 5 is implemented by a single transistor, which is triggered by an external triggering device, to switch between the on state and the off state. The external triggering device may be implemented by a single-chip microcomputer or an embedded device (e.g., Raspberry Pi hardware, Arduino hardware or the like), and is configured to be arbitrarily controlled to output a trigger signal for triggering the switch 5. However, implementation of the switch 5 is not limited to the disclosure herein and may vary in other embodiments.

In one embodiment, the switch 5 is configured to control the light source 31 in an intermittent way. That is to say, the switch 5 is configured to stay in the on state for a preset emitting time period and to allow the light source 31 to emit the laser beam to one of the acupuncture points during the preset emitting time period, to switch to the off state for a preset break time period to make the light source 31 cease to emit the laser beam, and to switch to the on state when the preset break time period has elapsed to allow the light source 31 to emit the laser beam to another one of the acupuncture points for the preset emitting time period. Further, during the off state, the adjusting unit 4 also orients the optical component 32 toward another one the acupuncture points. The preset emitting time period ranges from one to two seconds, and the preset break time period ranges from 0.1 to 0.3 seconds. Since the laser beam is not emitted continuously, power consumption of the system may be reduced, and a body part having no acupuncture point thereon would not be radiated by the laser beam while the manipulator 41 is orienting the optical component 32.

The user interface 6 is electrically connected to the image processing unit 2 and the adjusting unit 4. The user interface 6 is configured to allow user operation to input instructions and parameters with respect to the image processing unit 2 and the adjusting unit 4. It should be noted that according to different clinical conditions (e.g., the type of illness and its severity), the preset emitting time period can be adjusted by operating the user interface 6. The user interface 6 may be implemented by an independent touchscreen, or a touchscreen of a tablet, a personal computer, a notebook computer or any electronic device. However, implementations of the user interface 6 are not limited to what are disclosed herein and may vary in other embodiments.

For the purpose of explanation, an example of operating the system to perform laser acupuncture on the subject according to the disclosure is described below. In the example, the body part 9 of the subject to be treated using laser acupuncture is an arm of the subject.

Referring to FIGS. 2 and 5, a user operates the user interface 6 to input parameters to the system, wherein the parameters are exemplarily related to illness of the subject, laser intensity, a total operation time (i.e., a length of time of using the system to treat the illness), and so on. Thereafter, the image capturing device 21 captures an image of a top view of the arm (i.e., the body part 9) of the subject to serve as the target image 211. For the example shown in FIG. 5, the arm has three acupuncture points 90 (hereinafter respectively referred to as first, second and third acupuncture points 91-93 from left to right of FIG. 5). The processor 22 generates the control signal 221 based on the target image 211, and sends the control signal 221 to the controller 42 of the adjusting unit 4. In response to receipt of the control signal 221, the controller 42 controls the manipulator 41 to orient the optical component 32 based on the control signal 221 such that the laser beam emitted from the light source 31 can be directed to the first to third acupuncture 91-93 points one at a time (in any given sequence).

Specifically, at first, when the optical component 32 has been oriented for directing the laser beam to the first acupuncture point 91, the switch 5 operates in the on state for the preset emitting time period (e.g., one second) to allow the light source 31 to emit the laser beam to irradiate the first acupuncture point 91 for the preset emitting time period. In this way, treatment by using laser acupuncture on the first acupuncture point 91 is performed.

Subsequently, when the preset emitting time period has elapsed, the switch 5 immediately switches to the off state for the preset break time period (e.g., 0.1 seconds) to make the light source 31 cease to emit the laser beam. During the preset break time period, the adjusting unit 4 orients the optical component 32 for directing the laser beam to the second acupuncture point 92. Then, the switch 5 immediately switches from the off state to the on state when the preset break time period has elapsed.

Likewise, the switch 5 operates in the on state for the preset emitting time period to allow the light source 31 to emit the laser beam to irradiate the second acupuncture point 92 for the preset emitting time period, and then switches to the off state for the preset break time period to make the light source 31 cease to emit the laser beam. During the preset break time period, the adjusting unit 4 orients the optical component 32 for directing the laser beam to the third acupuncture point 93. The switch 5 then switches from the off state to the on state once the preset break time period has elapsed.

Then, the switch 5 operates in the on state for the preset emitting time period to allow the light source 31 to emit the laser beam to irradiate the third acupuncture point 93 for the preset emitting time period, and then switches to the off state for the preset break time period to make the light source 31 cease to emit the laser beam. During the preset break time period, the adjusting unit 4 orients the optical component 32 for directing the laser beam back to the first acupuncture point 91. The switch 5 switches from the off state to the on state when the preset break time period has elapsed to allow the light source 31 to emit the laser beam to irradiate the first acupuncture point 91 for the preset emitting time period, so on and so forth.

The above-mentioned procedure repeats until the total operation time has elapsed.

Referring to FIG. 6, a variant embodiment of the system according to the disclosure is illustrated. Since the variant embodiment is similar to the aforementioned embodiment, descriptions regarding identical components are not repeated, and only differences between the variant embodiment and the aforementioned embodiment will be explained in the following paragraphs for the sake of brevity.

In the variant embodiment, the light source 31 is mounted on the adjusting unit 4. The optical component 32 is a refracting lens that is placed in front of the light source 31 (the direction in which the light source 31 emits the laser beam is a forward or front direction, and the “front” of the light source 31 is defined from this perspective). Further, a distance between the light source 31 and the optical component 32 is shorter than that in the aforementioned embodiment. That is to say, compared with the aforementioned embodiment, the laser beam in the variant embodiment of FIG. 6 would travel in a relatively shorter path from the light source 31 to the optical component 32, and hence the laser beam in this embodiment would have relatively lower beam divergence and thereby relatively higher intensity. Consequently, quality of performance of laser acupuncture by using the system according to the variant embodiment may be relatively improved. Moreover, mounting both the light source 31 and the optical component 32 on the adjusting unit 4 may reduce space occupied by the system according to the disclosure. As a result, space utilization of a health facility in which the treatment using the system is performed may be increased.

To sum up, the system according to the disclosure utilizes the image capturing device 21 to capture an image of a body part, and utilizes the processor 22 to generate the control signal relating to acupuncture points on the body part based on the image thus captured. Afterwards, the system utilizes the light source 31 to emit a laser beam, and utilizes the adjusting unit 4 to orient, based on the control signal, the optical component 32 to direct the laser beam to the acupuncture points individually. In this way, for any acupuncture point on a body part of an individual, the system according to the disclosure is capable of automatically orienting the optical component 32 to direct the laser beam to the acupuncture point.

In the description above, for the purposes of explanation, numerous specific details have been set forth in order to provide a thorough understanding of the embodiments. It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A system for performing laser acupuncture on a plurality of acupuncture points on a body part of a subject, said system comprising:
a frame;
an image processing unit mounted on said frame, and including
an image capturing device that is configured to capture an image of the body part having the acupuncture points, and
a processor that is configured to generate, based on the image of the body part, a control signal that contains information related to positions respectively of the acupuncture points;
a laser unit mounted on said frame, spaced apart from said image processing unit, and including
a light source that is configured to emit a laser beam, and
an optical component that is located in a path, along which the laser beam travels, and that is configured to direct the laser beam; and
an adjusting unit mounted on said frame, electrically connected to said processor, interconnecting said optical component and said frame, and configured to receive the control signal, and to orient, based on the control signal, said optical component to direct the laser beam to the acupuncture points individually.

2. The system as claimed in claim 1, further comprising:
a switch electrically connected to said light source and said adjusting unit, and configured to switch between an on state where said switch enables said light source to emit the laser beam, and an off state where said switch disables said light source so said light source is unable to emit the laser beam.

3. The system as claimed in claim 2, wherein said switch is configured to control said light source in an intermittent way by repeatedly staying in the on state for a preset emitting time period to allow said light source to emit the laser beam to one of the acupuncture points during the preset emitting time period, switching to the off state for a preset break time period to make said light source cease to emit the laser beam, and switching to the on state when the preset break time period has elapsed.

4. The system as claimed in claim 3, wherein the preset emitting time period ranges from one to two seconds.

5. The system as claimed in claim 3, wherein the preset break time period ranges from 0.1 to 0.3 seconds.

6. The system as claimed in claim 3, wherein said adjusting unit is configured to orient said optical component when said switch operates in the off state.

7. The system as claimed in claim 1, wherein said optical component is a reflector.

8. The system as claimed in claim 1, wherein said optical component is a refracting lens.

9. The system as claimed in claim 1, further comprising:
a user interface electrically connected to said image processing unit and said adjusting unit, and configured to allow user operation to input instructions and parameters with respect to said image processing unit and said adjusting unit.

10. The system as claimed in claim 9, further comprising:
a power supply electrically connected to said image processing unit, said laser unit, said adjusting unit and said user interface, and configured to supply electricity thereto.

11. The system as claimed in claim 1, wherein said processor is configured to utilize a first artificial neural network (ANN) to add markers to the image of the body part respectively at the acupuncture points, to utilize a second ANN to obtain specific orientations according to the markers, and to generate the control signal based on the specific orientations thus obtained.

12. The system as claimed in claim 11, wherein the first ANN is established under generative adversarial network (GAN) framework, and is configured to identify positions respectively of the acupuncture points in an image of a body part of a subject.

13. The system as claimed in claim 12, wherein the first ANN includes a generator that is implemented by a U-Net, and a discriminator that is implemented by a Patch-GAN.

14. The system as claimed in claim 11, wherein the first ANN is trained by using techniques of paired image-to-image translation based on a plurality of pairs of training images.

15. The system as claimed in claim 14, wherein for each pair of training images, one image of the pair of training images contains a body part having no indication on where acupuncture points are located on the body part, and the other image of the pair of training images contains the same body part having indications respectively indicating where acupuncture points are located on the body part.
